(19) United States
(12) Patent Application Publication
(10) Pub. No.: US 2009/0288950 A1
(43) Pub. Date: Nov. 26, 2009

(54) LIQUID ELECTRO-MECHANICAL PRIME MOVER

(76) Inventor: Tihiro Ohkawa, La Jolla, CA (US)

Correspondence Address: NYDEGGER & ASSOCIATES, 348 OLIVE STREET, SAN DIEGO, CA, 92103, US

(21) Appl. No.: 12/123,837

(22) Filed: May 20, 2008

(51) Int. Cl. C07K 1/26

(52) U.S. Cl. 204/450; 204/600

(57) ABSTRACT

A liquid prime mover is provided that includes a support member for holding a contiguous liquid entity (e.g. a water droplet) having a defined dimension “d”. An ion permeable exchange membrane is mounted on the support member. Also, a positive electrode and a negative electrode are mounted on the support member with a distance between them. Importantly, the ion permeable membrane is positioned between the respective electrodes and the liquid entity. A voltage source is connected between the electrodes to establish an ion flow passing through the exchange membrane, and through the liquid entity when “d” overlays the distance between the electrodes, to thereby move the liquid entity on the support member.

### FIELD OF THE INVENTION

The present invention pertains generally to systems and methods for moving a small entity of a contiguous liquid, such as a water droplet, along a prescribed path. More particularly, the present invention pertains to systems and methods that move liquid entities by ohmic resistance. The present invention is particularly, but not exclusively, useful as a system or method for immobilizing one type ion (e.g. negative ion), while a current of the other type ion (e.g. positive ion) creates a motive force for the liquid entity.

### BACKGROUND OF THE INVENTION

As an electric current is passed through an electrolyte solution, the effect is to cause a chemical decomposition in the solution. The consequence of this decomposition is the neutralization of positive and negative ions at the electrodes; a phenomenon known as electrolysis.

During electrolysis, the respective densities of positive ions and negative ions are nearly equal and, since both positive and negative ions are present, the net force of the ions acting on the solution will be substantially zero. On the other hand, if one type ion (e.g. negative ion) is immobilized by other than electrical means, the other type ion (e.g. positive ion), will exert a force on the solution. Specifically, this force is due to ohmic resistance of the mobile ion as it moves through the liquid solution. As has been demonstrated, this resultant force can move the liquid.

For example, consider an arrangement wherein two ion permeable membranes are positioned in a liquid (e.g. water). In this arrangement, each membrane has a respective area “S”, and each membrane is permeable to a same type ion (e.g. a positive ion). Further, consider the membranes are located at a distance “L” from each other, and electrodes are positioned opposite the pair of membranes from each other. The volume flux “Γ” in the region between the two membranes, and the pump pressure “Δp” in this region can be mathematically expressed. Importantly, these expressions include operational parameters that are indicative of the fluid flow efficiency for the arrangement. In this context, for positive ions, it can be mathematically shown that:

Γ=[SK/2][jL/μ₊−Δp]

where: j is current density, K is the water permeability coefficient of the membranes, and μ₊ is the positive ion mobility in water. It needs to be appreciated, however, there are limitations for the above mathematical expression. These limitations concern the density of positive ions, and current density. Nevertheless, with these limitations accounted for, additional mathematical manipulations show that maximum pumping power “Pₚᵤₘₚ” occurs when:

Δp=jL/2 μ₊; and

Γ=[SK/4][jL/μ₊]

Functionally, the import of the mathematical expressions presented above is that an operational relationship can be established between the ion permeability of a membrane, and the electric current density that is necessary to generate a motive force on a liquid entity. More specifically, as noted above, when only ions of a same type (positive or negative) flow through a liquid, a resultant force is exerted on the liquid due to ohmic resistance. Under appropriate conditions, this force will cause the fluid to move in the direction of the ion flow. As implied above, the conditions for this to happen will be influenced by characteristics of the membranes that are used.

When considering the characteristics of a membrane, the generation of a force that will cause a fluid to flow depends primarily on how much ion momentum is lost in the membrane. This, in turn, will depend on the permeability of both the ions and the liquid (water) in the membrane. Typically, values of permeability for both ions and water in the membrane are small. This indicates the loss of ion momentum is due predominantly to their interaction with the membrane lattice and not with the water. The force due to the limited ion permeability is predominately on the membrane, not on water.

In light of the above, it is an object of the present invention to provide a liquid prime mover having a region of electrolyte solution that is bound at both ends by membranes permeable to the same species of ions, for movement of liquid through the region due to the ohmic resistance of permeable ions when an electric current passes through the region. Another object of the present invention is to provide a liquid prime mover that is easy to use, relatively simple to manufacture, and comparatively cost effective.

### SUMMARY OF THE INVENTION

In accordance with the present invention, a device for moving a liquid entity on a support member relies on the ohmic resistance that is generated in the liquid entity when a flow of ions passes through the entity. As envisioned for the present invention, either positive ions or negative ions can be used to provide the motive force for the liquid entity. In either case, an ion permeable exchange membrane(s) is(are) used to allow the flow of only one type ion (positive or negative) through a defined portion of the liquid entity. A proton exchange membrane can be used for this purpose when the solution is acidic. On the other hand, for saline solutions, membranes that are permeable to sodium or chlorine ions can be used. For purposes of the present invention, the liquid entity is contiguous in the defined portion, and the defined portion of the liquid entity has a dimension “d”. Typically, in the case of a droplet, the dimension “d” will be the diameter of the droplet. On the other hand, if the liquid entity lies on a trough, the dimension “d” can be a specified distance along the length of the liquid-filled trough.

Structurally, the device of the present invention includes a support member for holding the liquid entity as it is being moved. With different embodiments, the support member can be an open conduit (i.e. a “trench”), a closed conduit (i.e. a tube) or a flat plate. Regardless of its configuration, the support member will effectively establish a pathway for the liquid entity. Importantly, in each case an ion permeable exchange membrane is associated with the pathway on the support member. In all configurations, however, it is important that the defined portion of the liquid entity (i.e. the volume of liquid wherein the motive force is applied) must somehow be bounded by an ion permeable exchange membrane(s).

To establish a current path for the present invention, a positive electrode is positioned at a first location adjacent the membrane on the support member. And, a negative electrode is positioned at a second location adjacent the membrane on the support member. Importantly, both the positive electrode and the negative electrode are positioned opposite the membrane from the defined portion of the liquid entity. Thus, a current path is established that extends from an anode, through the membrane and into the defined portion of the liquid entity. The current path then continues from the defined portion and, again, through a membrane to the cathode. For all embodiments, the distance “L” (i.e. distance along the current path from membrane to membrane) must be less than the dimension “d” of the liquid entity. Stated differently, the liquid must be contiguous from membrane to membrane. The pumping action on water then occurs in the defined portion (i.e. the solution region that is between the membranes). The magnitude “f” of this pumping force per unit volume can be shown to be:

f=j/μ₊

where j is the current density and μ₊ is the mobility of the positive ion.

As will be appreciated by the skilled artisan, only two electrodes are required for the present invention. Nevertheless, for disclosure purposes, it will be appreciated that a plurality of electrodes can be employed and positioned on the support member. Further, when a plurality of electrodes is employed, a controller can be provided to selectively establish a particular electrode as either a positive electrode or a negative electrode.

In the operation of the prime mover of the present invention, a positive electrode and a negative electrode are simultaneously activated. This activation can be done selectively, but must occur when the dimension “d” of the contiguous liquid entity overlays or extends through the distance “L” between membrane surfaces. Recall, the activated electrodes and the liquid entity are on opposite sides of the ion permeable exchange membrane. Under these conditions, depending on the type of ion permeable membrane being used, only ions of the permeable type will flow in the liquid entity through the distance “L”. The resultant ohmic resistance then causes the liquid entity to move on the support member.

For a preferred embodiment of the present invention, the support member is a substantially fiat plate having a first side and a second side. In this embodiment, the ion exchange member is positioned against the first side of the plate. Further, a plurality of electrodes can be mounted in a matrix array on the second side of the plate. In this matrix array there are a plurality of rows and a plurality of columns. Due to the structural configuration of this embodiment, each electrode in a row will be substantially at the distance “L” from each adjacent electrode in the row. Also, each electrode in a column is substantially at the distance “L” from each adjacent electrode in the column. A controller can then be used to activate a voltage source and thereby selectively establish each electrode as a positive electrode or a negative electrode.

In a variation of the preferred embodiment of the present invention, the second side of the support member can be formed with an elongated open conduit (i.e. trench), with or without branches. In this embodiment the elongated conduit can be defined as having a first end and a second end, with at least one positive electrode positioned between the first end and the second end of the conduit. At least one negative electrode can then be positioned between the positive electrode and the second end of the conduit. Again, the controller can be used to activate the voltage source for moving the liquid entity along the conduit.

In an alternate preferred embodiment of the present invention, the conduit can be formed as a tubular structure having a wall formed with a lumen defining a longitudinal axis. In this embodiment, the ion permeable membrane is positioned against the wall of the conduit to surround the lumen of the conduit. Alternatively, the ion permeable membrane can include a first membrane that is positioned across the lumen of the conduit, and a second membrane that is positioned across the lumen of the conduit from the first membrane. In this case, both the first and the second membranes are located between the positive electrode and the negative electrode. For a variation of this embodiment, the cross-section of the conduit is rectangular shaped to establish a top panel, a bottom panel and opposed side walls. Also, the first and second membranes are rectangular shaped with respective side edges mounted on the opposing side walls of the conduit in a zigzag pattern, with a respective first end affixed to the bottom panel and a respective second end affixed to the top panel.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a closed-conduit, liquid prime mover in accordance with the present invention;

FIG. 2A is a cross-section view of a closed-conduit, two-membrane embodiment of the prime mover shown in FIG. 1, as seen along the line 2-2 in FIG. 1;

FIG. 2B is a cross-section view of a closed-conduit, single-membrane embodiment of the prime mover shown in FIG. 1, as seen along the line 2-2 in FIG. 1;

FIG. 3 is a perspective view of an open-conduit (i.e. trench) version of a liquid prime mover in accordance with the present invention;

FIG. 4 is a cross-section view of the prime mover shown in FIG. 3, as seen along the line 4-4 in FIG. 3;

FIG. 5 is a perspective view of an alternate embodiment of the closed-conduit prime mover in accordance with the present invention;

FIG. 6 is a cross-section view of the closed-conduit prime mover shown in FIG. 5, as seen along the line 6-6 in FIG. 5;

FIG. 7 is a flat plate version of a liquid prime mover in accordance with the present invention;

FIG. 8 is a bottom plan view of an electrode array for the flat plate prime mover as seen along the line 8-8 in FIG. 7; and

FIG. 9 is a cross section view of the flat plate prime mover as seen along the line 9-9 in FIG. 7.

### DESCRIPTION OF THE PREFERRED EMBODIMENTS

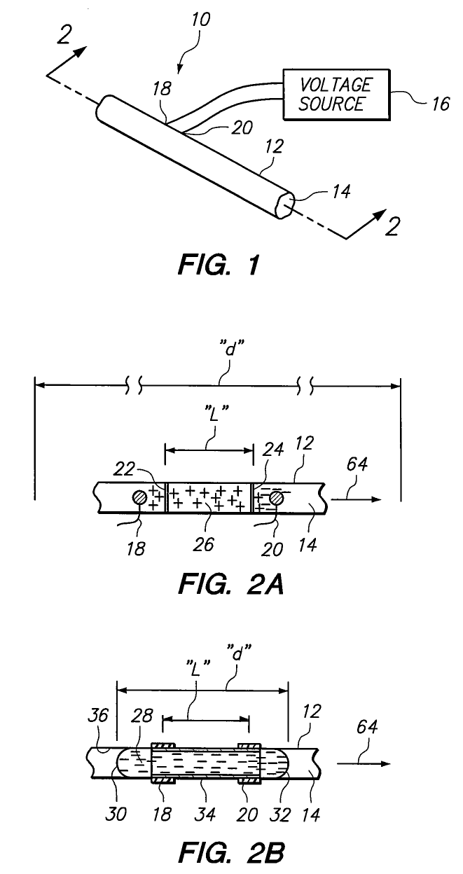

Referring initially to FIG. 1, a prime mover in accordance with the present invention is shown and is generally designated 10. In FIG. 1 it can be seen that the prime mover 10 involves a hollow tube 12 that is formed with an enclosed fluid channel 14. The prime mover 10 also includes a voltage source 16 that is connected with the tube 12 by two electrodes; a positive electrode 18 and a negative electrode 20. For the present invention, there can obviously be many more electrodes than the electrodes 18 and 20. Further, as envisioned for the present invention, the voltage source 16 can include a processor/controller (not shown) that can selectively establish or change the polarity of an electrode 18 or 20.

Possible internal structures for the prime mover 10 are best appreciated with reference to FIGS. 2A and 2B. In FIG. 2A, one embodiment of the prime mover 10 incorporates an ion permeable membrane 22 and an ion permeable membrane 24. For purposes of the present invention, the membranes 22 and 24 may be permeable to either positive or negative ions. Both membranes 22 and 24, however, need to be the same type membrane and, therefore, permeable to the same type ion. Preferably, the membranes 22 and 24 are each a so-called Proton Exchange Membrane (PEM), such as “Nafion” manufactured by the DuPont company. With such a structure, the prime mover 10 will immobilize negative ions and prevent them from entering the region 26 (i.e. the defined portion of a liquid entity 28) between the membranes 22 and 24. On the other hand, because they are permeable to the membranes 22 and 24, positive ions (e.g. protons) can pass through the region 26. And, because the membranes 22 and 24 are also permeable to liquid in the region 26, the ohmic resistance between the positive ions and the liquid will cause the liquid to flow through the region 26. As noted earlier, PEMs are preferably used with acidic solutions and membranes permeable to sodium and chlorine ions are used with saline solutions. Dimensionally, the embodiment of the prime mover 10 in FIG. 2A indicates that the liquid extends in the tube 12 through a dimension “d”. Further, FIG. 2A also shows that both membranes 22 and 24 are located between the electrodes 18 and 20 and are separated from each other by the distance “L”.

Another embodiment of the prime mover 10 is shown in FIG. 2B. In this case, the liquid in the fluid pathway (channel) 14 of tube 12 is shown as a contiguous liquid entity 28 having an end 30 and an end 32 that are at a dimension “d” from each other. Here, however, there is but one ion permeable membrane 34. As shown, the membrane 34 is positioned on the wall 36 of the tube 12. Importantly, the membrane 34 is between the liquid entity 28 and both of the respective electrodes 18 and 20. Stated differently, the liquid entity 28 extends over (i.e. overlays) both of the electrodes 18 and 20. Indeed, this dimensional relationship is required for the embodiment of prime mover 10 shown in FIG. 2A as well as for the embodiment shown in FIG. 2B. In any event, the current path from electrode 18 to electrode 20 will pass through the membrane 34 at different locations, and will pass through the liquid entity 28 between these locations (i.e. d>L).

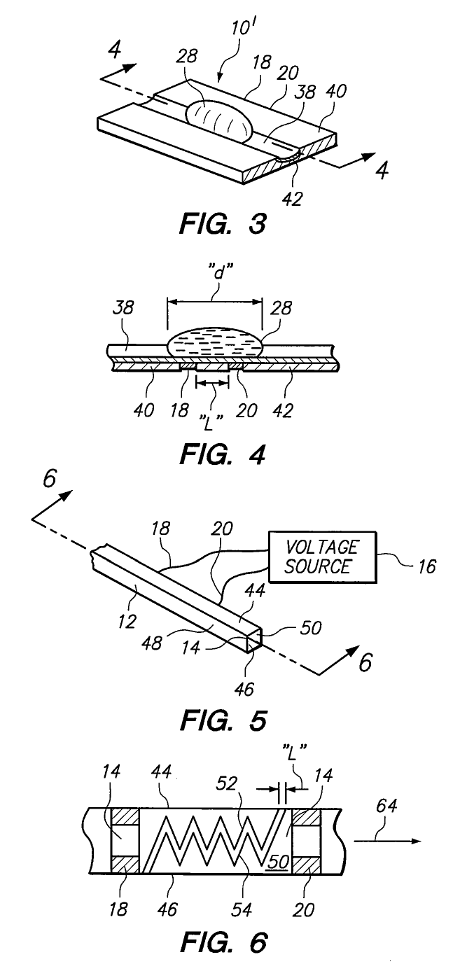

FIG. 3 shows a configuration for a prime mover 10′ that is formed with an open fluid pathway (channel) 38 (i.e. a trench or groove). More specifically, the open fluid pathway 38 is formed on the surface of a plate 40. For the prime mover 10′, an ion permeable membrane 42 provides a liner for the open fluid pathway 38. As shown in FIG. 4 the electrodes 18 and 20 are located under the open fluid pathway 38, to position the membrane 42 between the electrodes 18 and 20 and the contiguous liquid entity 28 that will travel along the open fluid pathway 38. In all essential operational respects, the prime mover 10′ functions substantially in the same way as the embodiment of prime mover 10 shown in FIG. 2B. As before, the distance “L” between locations on the membrane 42 needs to be less than the dimension “d” of the liquid entity 28.

Another embodiment of the prime mover 10 is shown in FIG. 5 wherein the enclosed fluid channel 14 is rectangular shaped. As shown, the rectangular shaped fluid channel 14 is bounded by a top panel 44, a bottom panel 46 and opposed side walls 48 and 50. The usefulness of such a configuration is perhaps best appreciated with reference to FIG. 6. There it is indicated that the opposed side walls 48 and 50 can be used to support an ion permeable membrane 52 and an ion permeable membrane 54. In this case, the membranes 52 and 54 are directly separated from each other by the distance “L”, and both membranes 52 and 54 are configured with a zigzag pattern. For this embodiment of the present invention, it is to be understood that a liquid (i.e. water) will fill the enclosed fluid channel 14. And, in this case the distance “L” is shown as being the separation distance between the membranes 52 and 54. Importantly, regardless whether the liquid entity 28 is a water droplet on a plate, or extends through the tube 12, the electrodes 18, 20 must always be at least at a distance greater than “L” from each other. And, “d” must also be greater than “L”.

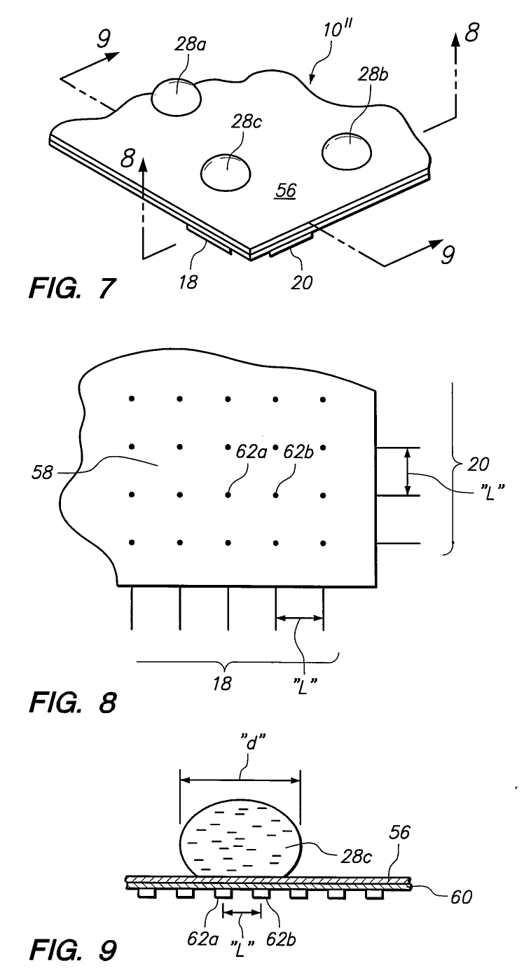

In yet another embodiment of the present invention, a prime mover 10″ is shown in FIG. 7. In this case a flat plate 56 is being used as a support member for a plurality of liquid entities 28 (the entities 28a, b and c are only exemplary). As best appreciated with cross reference to FIG. 8, an electrode array 58 is positioned on the underside of the flat plate 56, with an ion permeable membrane 60 located between the plate 56 and the electrode array 58. For the prime mover 10″, individual electrodes 62 in the array 58 can be established as either a positive electrode 18 or a negative electrode 20. For example, the electrode 62a may be positive while the electrode 62b is negative, and vice versa. The point is that the electrodes 62 can be programmed to be of a selected potential depending on the desired movement of the liquid entity 28 over the plate 56.

In the operation of the prime mover 10 (N.B. the prime movers 10′ and 10″ will operate the same way) a liquid entity 28 (or a liquid body) is positioned so that a dimension “d” of the liquid entity 28 simultaneously overlies two electrodes (e.g. electrodes 18 and 20 [see FIG. 2A]). In this example, illustrated in FIG. 2A, when a voltage potential is applied to the electrodes 18 and 20, negative ions “−” will be immobilized from entering the region 26 by the permeable membranes 22 and 24. Protons (i.e. positive ions “+”), however, will move through the region 26 because they are permeable to the membranes 22 and 24. The resultant ohmic resistance will generate a force that causes the liquid in the prime mover 10 to move in the direction of the arrow 64.

While the particular Liquid Electro-Mechanical Prime Mover as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A prime mover for a liquid medium which comprises:
a support member for holding a portion of the liquid medium as a contiguous entity having a dimension “d”;
a positive electrode mounted on the support member;
a negative electrode mounted on the support member;
at least one ion permeable exchange membrane positioned on the support member between the liquid entity and the positive electrode, and between the liquid entity and the negative electrode to immobilize a first type ion; and
a voltage source connected to the positive electrode and to the negative electrode to establish a flow of a second type ion therebetween, with the ion flow passing through the liquid entity to move the liquid entity on the support member.

2. A prime mover as recited in claim 1 wherein the support member is an elongated conduit having a first end and a second end, with the conduit defining a fluid pathway therebetween, and wherein the positive electrode is positioned between the first end and the second end of the conduit, with the negative electrode positioned between the positive electrode and the second end of the conduit.

3. A prime mover as recited in claim 2 wherein the conduit is a tubular structure having a wall and is formed with a lumen defining a longitudinal axis, and the ion permeable membrane is positioned against the wall of the conduit to surround the lumen of the conduit.

4. A prime mover as recited in claim 3 wherein the ion permeable membrane is a first membrane positioned across the lumen of the conduit, and the prime mover further comprises a second ion permeable membrane positioned across the lumen of the conduit at the axial distance “L” from the first membrane, with both the first and the second membranes located between the positive electrode and the negative electrode.

5. A prime mover as recited in claim 4 wherein the first membrane and the second membrane are positioned substantially perpendicular to the axis of the conduit.

6. A prime mover as recited in claim 5 wherein the positive electrode and the negative electrode are positioned in the lumen of the conduit.

7. A prime mover as recited in claim 4 wherein the cross-section of the conduit is rectangular to establish a top panel, a bottom panel and opposed side walls, and wherein the first and the second membranes are rectangular shaped with respective side edges mounted on the opposing side walls of the conduit in a zigzag pattern, with a respective first end affixed to the bottom panel and a respective second end affixed to the top panel.

8. A prime mover as recited in claim 1 wherein the support member is a substantially flat plate having a first side and a second side, wherein the ion exchange member is positioned against the first side of the plate and the prime mover further comprises:
a plurality of electrodes mounted in a matrix array, with the matrix array having a plurality of rows and a plurality of columns, wherein each electrode in a row is at the distance “L” from each adjacent electrode in the row, and each electrode in a column is at the distance “L” from each adjacent electrode in the column; and
a controller for selectively establishing each electrode as a positive electrode and, alternatively, as a negative electrode.

9. A prime mover as recited in claim 1 wherein the distance “L” is approximately one millimeter.

10. A prime mover as recited in claim 1 wherein the ion permeable exchange membrane is a Proton Exchange Membrane (PEM) and the ion flow is a proton flow.

11. A liquid prime mover which comprises:
an ion permeable exchange membrane;
a positive electrode positioned at a first location adjacent the membrane;
a negative electrode positioned at a second location adjacent the membrane;
a contiguous liquid entity positioned against the membrane opposite the positive electrode and opposite the negative electrode across the membrane; and
a voltage source connected to the positive electrode and to the negative electrode to establish an ion flow therebetween, with the ion flow passing through the exchange membrane for interaction with the liquid entity, to move the liquid entity on the support member.

12. A prime mover as recited in claim 11 wherein the ion permeable membrane is positioned on a support member.

13. A prime mover as recited in claim 12 wherein the support member is an elongated conduit having a first end and a second end, with the conduit defining a fluid pathway therebetween, and wherein the positive electrode is positioned between the first end and the second end of the conduit, with the negative electrode positioned between the positive electrode and the second end of the conduit.

14. A prime mover as recited in claim 12 wherein the conduit is a tubular structure having a wall and is formed with a lumen defining a longitudinal axis, and the ion permeable membrane is positioned against the wall of the conduit to surround the lumen of the conduit.

15. A prime mover as recited in claim 12 wherein the ion permeable membrane is a first membrane positioned across the lumen of the conduit, and the prime mover further comprises a second ion permeable membrane positioned across the lumen of the conduit at the axial distance “L” from the first membrane, with both the first and the second membranes located between the positive electrode and the negative electrode.

16. A prime mover as recited in claim 11 wherein the ion permeable exchange membrane is a Proton Exchange Membrane (PEM).

17. A prime mover as recited in claim 11 wherein the ion permeable membrane is permeable to sodium and chlorine ions.

18. A prime mover as recited in claim 11 wherein the liquid entity is a water droplet.

19. A method for moving a contiguous liquid entity having a dimension “d” which comprises the steps of:
providing a prime mover comprising an ion permeable exchange membrane, a positive electrode positioned at a first location adjacent the membrane, a negative electrode positioned at a second location adjacent the membrane, and a voltage source connected to the positive electrode and to the negative electrode to establish an ion flow therebetween, with the ion flow passing through the exchange membrane for interaction with the liquid entity; and
operating the voltage source to selectively activate the positive and negative electrodes, to move the liquid entity on the support member.

20. A method as recited in claim 19 wherein the liquid entity is a water droplet.
